(19) United States
(12) Patent Application Publication
(10) Pub. No.: US 2025/0262133 A1
(43) Pub. Date: Aug. 21, 2025

(54) COSMETIC COMPOSITION OF POLYOL-IN-OIL FORMULATION

(71) Applicant: AMOREPACIFIC CORPORATION, Seoul (KR)

(72) Inventors: Yujin JEONG, Yongin-si (KR); Hajin JUNG, Yongin-si (KR)

(73) Assignee: AMOREPACIFIC CORPORATION, Seoul (KR)

(21) Appl. No.: 19/049,212

(22) Filed: Feb. 10, 2025

(30) Foreign Application Priority Data
Feb. 15, 2024 (KR) 10-2024-0021568

(51) Int. Cl. A61K 8/04; A61K 8/02; A61K 8/25; A61K 8/34; A61K 8/58; A61K 8/92; A61Q 1/06

(52) U.S. Cl. CPC A61K 8/04; A61K 8/0229; A61K 8/25; A61K 8/345; A61K 8/585; A61K 8/922; A61Q 1/06

(57) ABSTRACT

The present disclosure discloses a cosmetic composition of a polyol-in-oil formulation. The composition includes wax, oil, polyol, porous powder, and an inorganic thickening agent. The composition stabilizes a high content of polyol components in an anhydrous polyol-in-oil formulation using a minimal amount of emulsifier. The composition provides the effect of significantly improving sweating phenomena in formulations using natural wax and improving the dispersibility issue of pigment layer separation at high temperatures.

### CROSS REFERENCE TO RELATED APPLICATION

The present application claims priority to Korean Patent Application No. 10-2024-0021568, filed Feb. 15, 2024, the entire contents of which are hereby incorporated by this reference.

### BACKGROUND OF THE INVENTION

### Field of the Invention

The present disclosure discloses a cosmetic composition of a polyol-in-oil formulation.

### Description of the Related Art

Lipsticks are primarily composed of oil-dispersion formulations made of substances such as waxes, oils, and powders, which are hydrophobic or lipophilic, helping to prevent moisture evaporation from the lips. However, when a large amount of occlusive agent is used, it results in a heavy and sticky feeling in use, which is a drawback. In addition, there is a limitation in moisture retention, and prolonged use may cause dryness, requiring frequent reapplication.

Glycerin and butylene glycol, which are representative polyol components, are widely used humectants for skin moisturization. Among them, glycerin, with three hydroxyl groups (—OH) in its molecular structure, has a strong property of attracting moisture, is non-volatile, and has excellent moisturizing ability. It also provides a refresh feeling in use compared to oils.

However, when a large amount of hydrophilic polyols is applied to an oil-dispersion formulation, there are issues with physical stability, such as formulation separation or severe sweating on the surface, due to differences in compatibility. This results in decreased marketability and makes commercialization difficult. Sweating in stick products is not only visually unappealing but also causes issues during use, as the sweating components on the surface make it slippery and prevent proper application. Therefore, improvement is necessary.

Research has been conducted to stabilize polyols in stick formulations by mixing waxes, oils, and polyols with emulsifiers to manufacture polyol-in-oil formulations. However, to stabilize a high content of polyols, emulsifiers with high content, which raise concerns about skin irritation, had to be used. Additionally, severe sweating at both room temperature and high temperatures remained difficult to improve. In particular, when the stick is manufactured with a low hardness to provide a smooth, melting feeling in use during application, the sweating phenomenon becomes more pronounced, which limits the ability to achieve an excellent feeling in use.

Further, in case of lipsticks, the use of a large amount of colorant is essential for color expression on the lips. However, the polyol-in-oil formulation has low viscosity at high temperatures, and when stored in a high-temperature oven around 90° C. for extended periods to mold the lipstick, there is an issue with pigment layer separation. Additionally, when colorant is included, it affects the wax crystal structure, causing the sweating phenomenon to be much more pronounced compared to colorless formulations.

Recently, as consumer concerns about the safety of petroleum-based synthetic raw materials used in cosmetics and interest in the environment have risen, there has been a trend toward developing stick-type cosmetics using plant-derived waxes instead of petroleum-based synthetic waxes, which have mainly been used in lipstick manufacturing. However, when plant-derived waxes are used instead of synthetic waxes, there is a disadvantage in that the sweating phenomenon becomes more pronounced due to differences in crystal structure formation caused by the characteristics of the raw materials.

Therefore, there is a need to improve the drawbacks in conventional stick-type polyol-in-oil cosmetic compositions, such as sweating issues, poor feeling in use, and inadequate pigment dispersibility, which make commercialization difficult.

### SUMMARY OF THE INVENTION

In one aspect, the present disclosure is directed to providing a cosmetic composition of an polyol-in-oil formulation.

In one aspect, the present disclosure provides a cosmetic composition of a polyol-in-oil formulation comprising wax, oil, polyol, porous powder, and an inorganic thickening agent.

In an exemplary embodiment, the wax may include sunflower seed wax.

In an exemplary embodiment, the polyol may include glycerin.

In an exemplary embodiment, the polyol may further include one or more selected from the group consisting of butylene glycol, diglycerin, and propylene glycol.

In an exemplary embodiment, the porous powder may include silica, silica silylate, silica dimethyl silylate, silica/dimethicone, silica/methicone, methyl methacrylate crosspolymer, or mixtures thereof.

In an exemplary embodiment, the inorganic thickening agent may include one or more selected from the group consisting of hectorite and bentonite.

In an exemplary embodiment, the composition may include 0.1 to 15 wt % of wax, 30 to 70 wt % of oil, 5 to 25 wt % of polyol, 0.5 to 6 wt % of porous powder, and 0.1 to 3 wt % of inorganic thickening agent, based on the total weight of the composition.

In an exemplary embodiment, the polyol and the porous powder may be mixed in a weight ratio of 4 to 20:1.

In an exemplary embodiment, the composition may further include 0.1 to 10 wt % of pigment, based on the total weight of the composition.

In an exemplary embodiment, the composition may further include 0.1 to 5 wt % of emulsifier, based on the total weight of the composition.

In an exemplary embodiment, the composition may further include 5 to 25 wt % of emollient, based on the total weight of the composition.

In an exemplary embodiment, the mixed weight of the polyol and emollient may be 30 to 40 wt %, based on the total weight of the composition.

In an exemplary embodiment, the composition may be a lip makeup cosmetic composition.

In an exemplary embodiment, the composition may be a cosmetic composition in a stick formulation.

In an exemplary embodiment, the composition may be an anhydrous formulation.

In one aspect, the technology disclosed in the present disclosure has the effect of providing a cosmetic composition of an polyol-in-oil formulation.

In an exemplary embodiment, the composition stabilizes a high content of polyol components in an polyol-in-oil formulation using a minimal amount of emulsifier. In formulations using natural wax, the composition provides the effect of significantly improving sweating phenomena and feeling in use, as well as improving the dispersibility issue of pigment layer separation at high temperatures.

### BRIEF DESCRIPTION OF THE DRAWINGS

FIG. 1 illustrates a photograph taken after storing the stick formulation according to an embodiment at a high temperature of 45° C., visually confirming the sweating phenomenon (left: formulation of 1-2, right: formulation of 1-3).

FIG. 2 illustrates a photograph taken after storing the formulation of 2-1, manufactured in an embodiment, at a high temperature of 45° C., visually confirming the sweating phenomenon.

FIG. 3 illustrates the results of skin moisture level measurements over time for the stick formulation according to an experimental example.

FIG. 4 illustrates the results of moisture increase and decrease rate measurements over time for the stick formulation according to an experimental example.

### DETAILED DESCRIPTION OF THE INVENTION

Hereinafter, the present disclosure will be described in detail.

In one aspect, the present disclosure provides a cosmetic composition of a polyol-in-oil formulation comprising wax, oil, polyol, porous powder, and an inorganic thickening agent.

Polyols have excellent properties, such as outstanding moisturizing ability and providing a refresh feeling in use compared to oils. However, when a large amount of polyol is applied to an oil-dispersion formulation, there is an issue where the formulation separates or severe sweating occurs on the surface, making commercialization difficult. The present disclosure has the effect of significantly improving the sweating phenomenon that easily occurs in conventional polyol-in-oil cosmetics and solving the dispersibility issue where pigment layers separate at high temperatures, thereby providing a cosmetic composition of a polyol-in-oil formulation with excellent formulation stability, enhanced moisturizing ability, and improved feeling in use. In addition, in a formulation using natural wax, the present disclosure has the effect of providing a cosmetic composition of a polyol-in-oil formulation that stabilizes a high content, such as 5 wt % or more, 10 wt % or more, 15 wt % or more, or 20 wt % or more of polyols.

In an exemplary embodiment, the wax may include sunflower seed (Helianthus anmius seed) wax. In the cosmetic composition of a polyol-in-oil formulation according to the present disclosure, sunflower seed wax has excellent compatibility with various types of oils, forms good hardness, and provides the excellent effect of a smooth application without a stiff feeling in use.

In an exemplary embodiment, the wax may further include one or more selected from the group consisting of rice bran wax, candelilla wax, and carnauba wax.

In an exemplary embodiment, the content of the wax may be 0.1 to 15 wt %, based on the total weight of the composition, and preferably 0.1 to 10 wt %. In another exemplary embodiment, the content of the wax may be 0.1 wt % or more, 0.5 wt % or more, 1 wt % or more, 2 wt % or more, 3 wt % or more, 4 wt % or more, 5 wt % or more, 6 wt % or more, 7 wt % or more, 8 wt % or more, 9 wt % or more, or 10 wt % or more, and 15 wt % or less, 14 wt % or less, 13 wt % or less, 12 wt % or less, 11 wt % or less, or 10 wt % or less, based on the total weight of the composition.

In an exemplary embodiment, the oil may include one or more of: ester-based oils selected from the group consisting of hexyl laurate, dicaprylyl carbonate, diisostearyl malate, butylene glycol dicaprylate/dicaprate, cetyl ethylhexanoate, triethylhexanoin, dicetearyl dimer dilinoleate, caprylic/capric triglyceride, and pentaerythrityl tetraisostearate; hydrocarbon-based oils selected from the group consisting of polybutene, hydrogenated polyisobutene, and phytosqualane; or silicone-based oils selected from the group consisting of phenyl trimethicone and dimethicone.

In an exemplary embodiment, the content of the oil may be 30 to 70 wt %, based on the total weight of the composition. In another exemplary embodiment, the content of the oil may be 30 wt % or more, 35 wt % or more, 40 wt % or more, 45 wt % or more, or 50 wt % or more, and 70 wt % or less, 65 wt % or less, 60 wt % or less, 55 wt % or less, 50 wt % or less, 45 wt % or less, or 40 wt % or less, based on the total weight of the composition.

In an exemplary embodiment, the polyol may include glycerin. In case of conventional stick-type water-in-oil lip makeup compositions that include water to provide moisture, water rapidly evaporates at room temperature, causing the stick to shrink and deform over time. This necessitates the use of special sealed containers, and after application to the lips, the evaporation of moisture leads to an increase in dryness, which is a disadvantage. The present disclosure has the effect of providing a cosmetic composition of an anhydrous polyol-in-oil formulation using glycerin as the polyol, which can improve these drawbacks.

In an exemplary embodiment, the polyol may further include one or more selected from the group consisting of butylene glycol, diglycerin, and propylene glycol.

In an exemplary embodiment, the content of the polyol may be 5 wt % or more, 10 wt % or more, 15 wt % or more, or 20 wt % or more, based on the total weight of the composition.

In another exemplary embodiment, the content of the polyol may be 5 to 25 wt %, or for improving the formulation stability and feeling in use of the polyol-in-oil formulation including natural wax, it may preferably be 10 to 25 wt % or 10 to 20 wt %, based on the total weight of the composition. In another exemplary embodiment, the content of the polyol may be 5 wt % or more, 7 wt % or more, 10 wt % or more, 12 wt % or more, 15 wt % or more, 17 wt % or more, or 20 wt % or more, and 25 wt % or less, 22 wt % or less, 20 wt % or less, 17 wt % or less, or 15 wt % or less, based on the total weight of the composition.

In an exemplary embodiment, the porous powder may include silica (AEROSIL® 200, Evonik), silica silylate (AEROSIL® 812S, AEROSIL® 202, Evonik), silica dimethyl silylate (HDK® H15, HDK® H18, WACKER), silica/dimethicone (silica surface-treated with dimethicone), silica/methicone (silica surface-treated with methicone), methyl methacrylate crosspolymer, or mixtures thereof. Preferably, the porous powder is silica dimethyl silylate, which may show excellent effects in inhibiting sweating.

In an exemplary embodiment, the content of the porous powder may be 0.5 to 6 wt %, and preferably 1 to 5 wt % or 1 to 2 wt %, based on the total weight of the composition, in terms of inhibiting sweating and enhancing feeling in use. In another exemplary embodiment, the content of the porous powder may be 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, or 5 wt % or more, and 6 wt % or less, 5.5 wt % or less, 5 wt % or less, 4.5 wt % or less, 4 wt % or less, 3.5 wt % or less, 3 wt % or less, 2.5 wt % or less, or 2 wt % or less, based on the total weight of the composition.

In an exemplary embodiment, the polyol and porous powder may be mixed in a weight ratio of 4 to 20:1, 4 to 15:1, or 4 to 10:1 to improve the formulation stability and feeling in use of the polyol-in-oil formulation including natural wax.

In an exemplary embodiment, the wax and porous powder may be mixed in a weight ratio of 1:0.1 to 0.5, 1:0.2 to 0.5, or 1:0.3 to 0.4 to improve the formulation stability and feeling in use of the polyol-in-oil formulation including natural wax.

In an exemplary embodiment, the inorganic thickening agent may include one or more selected from the group consisting of hectorite and bentonite. The hectorite and bentonite inorganic thickening agents, when added in small amounts, increase the viscosity at high temperatures, helping with pigment dispersion in the polyol-in-oil formulation. They also prevent the contents from separating at high temperatures for extended periods after manufacturing, thereby facilitating the stick molding process. When the hectorite and bentonite inorganic thickening agents are not added, the pigment may not disperse well, leading to the separate formation of the pigment layer.

In an exemplary embodiment, the content of the inorganic thickening agent may be 0.1 to 3 wt %, based on the total weight of the composition. In another exemplary embodiment, the content of the inorganic thickening agent may be 0.1 wt % or more, 0.5 wt % or more, 1 wt % or more, 1.5 wt % or more, or 2 wt % or more, and 3 wt % or less, 2.5 wt % or less, 2 wt % or less, 1.5 wt % or less, or 1 wt % or less, based on the total weight of the composition.

In an exemplary embodiment, the composition may further include a pigment.

In an exemplary embodiment, the composition may further include 0.1 to 10 wt % of pigment, based on the total weight of the composition. In another exemplary embodiment, the composition may further include a pigment in an amount of 0.1 wt % or more, 0.5 wt % or more, 1 wt % or more, 1.5 wt % or more, 2 wt % or more, 2.5 wt % or more, or 3 wt % or more, and 10 wt % or less, 9.5 wt % or less, 9 wt % or less, 8.5 wt % or less, 8 wt % or less, 7.5 wt % or less, 7 wt % or less, 6.5 wt % or less, 6 wt % or less, 5.5 wt % or less, 5 wt % or less, 4.5 wt % or less, 4 wt % or less, 3.5 wt % or less, or 3 wt % or less, based on the total weight of the composition.

The pigment refers to a substance that does not dissolve in solvents such as water or oil, but instead disperses therein. The pigment may refer to a powdery colorant that does not dissolve and remains as a powder in the solvent and attaches to the surface of fibers or other materials. In an exemplary embodiment, the pigment may be broadly classified into organic pigments and inorganic pigments. The organic pigment refers to a pigment in which an organic compound is the main component of the colorant, including lake pigments. The inorganic pigment refers to a pigment that is chemically inorganic. In an exemplary embodiment, the inorganic pigment may include one or more selected from the group consisting of white pigments, color pigments, extender pigments, and pearl pigments.

In an exemplary embodiment, the white pigment may include one or more selected from the group consisting of titanium dioxide, zinc oxide, zinc sulfide, and barium sulfate.

In an exemplary embodiment, the extender pigment may include one or more selected from the group consisting of talc, mica, sericite, kaolin, boron nitride, silica, alumina, barium silicate, zeolite, barium sulfate, clay, kaolin, bentonite, calcium carbonate, and magnesium carbonate.

In an exemplary embodiment, the pigment may include pigments commonly used in cosmetics, for example, inorganic powders such as muscovite, synthetic mica, sericite, kaolin, titanium dioxide, titanium oxide-coated mica, aluminum powder, iron oxide yellow, iron oxide black, carbon black, barium sulfate, silica, and zeolite, as well as organic powders such as nylon, PMMA, polystyrene powder, polyethylene powder, and cellulose powder.

In an exemplary embodiment, the pigment may be organic pigments of barium or aluminum lakes, pearl pigments, etc., such as Yellow No. 203, Yellow No. 4, Yellow No. 5, Yellow No. 201, Yellow No. 204, Red No. 103, Red No. 201, Red No. 202, Red No. 218, Red No. 220, Red No. 223, Red No. 226, Red No. 227, Blue No. 1, Blue No. 2, etc.

In an exemplary embodiment, the composition may further include an emulsifier.

In an exemplary embodiment, the composition may further include an emulsifier in an amount of 5 wt % or less, 4.5 wt % or less, 4 wt % or less, 3.5 wt % or less, 3 wt % or less, 2.5 wt % or less, 2 wt % or less, 1.5 wt % or less, 1 wt % or less, 0.5 wt % or less, 0.1 wt % or less, 0.05 wt % or less, or 0.001 wt % or less, based on the total weight of the composition. The present disclosure provides the effect of stabilizing a high content of polyol components in a polyol-in-oil formulation by using a minimal amount of emulsifier.

In another exemplary embodiment, the composition may further include an emulsifier in an amount of 0.1 to 5 wt % or 1 to 5 wt %, based on the total weight of the composition. In another exemplary embodiment, the composition may further include an emulsifier in an amount of 0.1 wt % or more, 0.5 wt % or more, 1 wt % or more, 1.5 wt % or more, 2 wt % or more, 2.5 wt % or more, or 3 wt % or more, and 5 wt % or less, 4.5 wt % or less, 4 wt % or less, 3.5 wt % or less, or 3 wt % or less, based on the total weight of the composition.

In an exemplary embodiment, the emulsifier may be a nonionic emulsifier having a hydrophilic-lipophilic balance (HLB) value of 3 to 6. Preferably, Cetyl PEG/PPG-10/1 Dimethicone component (ABIL EM 90, EVONIK company) with high emulsifying power and a wide range of oil compatibility may be used at approximately 3 wt %.

In an exemplary embodiment, the composition may further include an emollient.

In an exemplary embodiment, the composition may further include 5 to 25 wt % of emollient, based on the total weight of the composition. In another exemplary embodiment, the composition may further include an emollient in an amount of 5 wt % or more, 10 wt % or more, 15 wt % or more, or 20 wt % or more, and 25 wt % or less, 20 wt % or less, 15 wt % or less, or 10 wt % or less, based on the total weight of the composition.

In an exemplary embodiment, the emollient may have a viscosity of 300 cP or more, 300 to 1,300 cP, or 300 to 1,000 cP at 25° C.

In an exemplary embodiment, the emollient may include polyglyceryl-2 triisostearate.

In an exemplary embodiment, the mixed weight of the polyol and emollient may be 30 to 40 wt %, based on the total weight of the composition.

In an exemplary embodiment, the composition may be a lip makeup cosmetic composition.

In an exemplary embodiment, the composition may be a cosmetic composition in a stick formulation.

In an exemplary embodiment, the composition may be an anhydrous formulation.

In an exemplary embodiment, in addition to the above components, the composition may further include components commonly used in cosmetic formulations, for example, emulsifiers other than the above, powders, polymer compounds, moisturizers, fragrances, antioxidants, preservatives, and active components that are suitably formulated in appropriate amounts that do not impair the effects described in the present specification.

Hereinafter, the present disclosure will be described in further detail through examples. These embodiments are just illustrative of the present disclosure, and it is apparent to one of ordinary skill in the art that the scope of the present disclosure is not to be interpreted as limited by these embodiments.

### EXAMPLE 1

The respective components, excluding the polyol, were placed into a container according to the formulation in Table 1 below, heated to 90° C., and thoroughly melted. Then, they were dispersed using a homo mixer at 2,000 rpm for 20 minutes. Subsequently, the polyol was added, and further emulsification was carried out. The degassed content at 90° C. was poured into a back filling type container and then cooled to mold the stick-type formulation.

A solid wax raw material with a melting point of approximately 80° C. was used for the synthetic wax.

Polyglyceryl-2 triisostearate is a high-viscosity oil-based emollient that is suitable for wetting pigments, thereby improving the dispersibility of the pigments. Polyglyceryl-2 triisostearate has a viscosity (400 cP at 25° C.) similar to that of glycerin (954 cP at 25° C.), and its content was adjusted according to the change in polyol content.

Methyl hydrogenated rosinate, which is a high-viscosity rosin component derived from pine resin, was added to provide gloss and adhesion.

Hydrogenated poly(C6-14 olefin) is a low-viscosity oil that provides a light feeling in use and silky application, while diisostearyl malate is a high-viscosity oil with excellent gloss, adding shine to the lipstick.

The hardness, sweating phenomenon, and feeling in use of each formulation manufactured were evaluated.

The hardness was measured and evaluated using a rheometer (CR-500DX-L, Sun Scientific Co., Ltd, Japan) at room temperature to measure and evaluate the cutting hardness.

The sweating phenomenon was evaluated visually after storing the sample in a sealed temperature-controlled chamber at 45° C. for 4 hours. When an oil was deposited on the entire areas of the stick, indicating significant sweating, it was marked as “O.” When only a small amount of oil appeared on some but not entire areas of the stick, with very low levels of sweating, it was marked as “Δ.” When no sweating phenomenon was observed on the entire areas of the stick, it was marked as “X.”

The feeling in use was evaluated with a subjective questionnaire focusing on the spreadability of each stick formulation, using five women in their 30s as participants.

TABLE 1

| | Components | 1-1 | 1-2 | 1-3 | 1-4 | 1-5 | 1-6 | 1-7 |
|---|---|---|---|---|---|---|---|---|
| 1 | Synthetic wax | 10 | 10 | 10 | | | | |
| 2 | Sunflower seed wax | | | | 10 | | | |
| 3 | Rice bran wax | | | | | 10 | | |
| 4 | Candelilla wax | | | | | | 10 | |
| 5 | Carnauba wax | | | | | | | 10 |
| 6 | Polyglyceryl-2 triisostearate | 30 | 5 | 5 | 5 | 5 | 5 | 5 |
| 7 | Methyl hydrogenated rosinate | 10 | 10 | 10 | 10 | 10 | 10 | 10 |
| 8 | Hydrogenated poly(C6-14 Olefin) | 26 | 26 | 26 | 26 | 26 | 26 | 26 |
| 9 | Diisostearyl malate | 15 | 15 | 15 | 15 | 15 | 15 | 15 |
| 10 | Cetyl PEG/PPG-10/1 | 3 | 3 | 3 | 3 | 3 | 3 | 3 |
| | dimethicone*pentaerythrityl | | | | | | | |
| | tetra-di-t-butyl | | | | | | | |
| | hydroxyhydrocinnamate | | | | | | | |
| 11 | Disteardimonium hectorite | 1 | 1 | 1 | 1 | 1 | 1 | 1 |
| 12 | Pigments | 5 | 5 | 5 | 5 | 5 | 5 | 5 |
| 13 | Glycerin | | 25 | | 25 | 25 | 25 | 25 |
| 15 | Butylene Glycol | | | 25 | | | | |

| | | | | | | | |
|---|---|---|---|---|---|---|---|
| Hardness (gf/mm²) | 74 | 68 | 58 | 65 | 48 | 32 | — |
| 45° C. sweating | X | Δ | ◯ | ◯ | ◯ | ◯ | — |

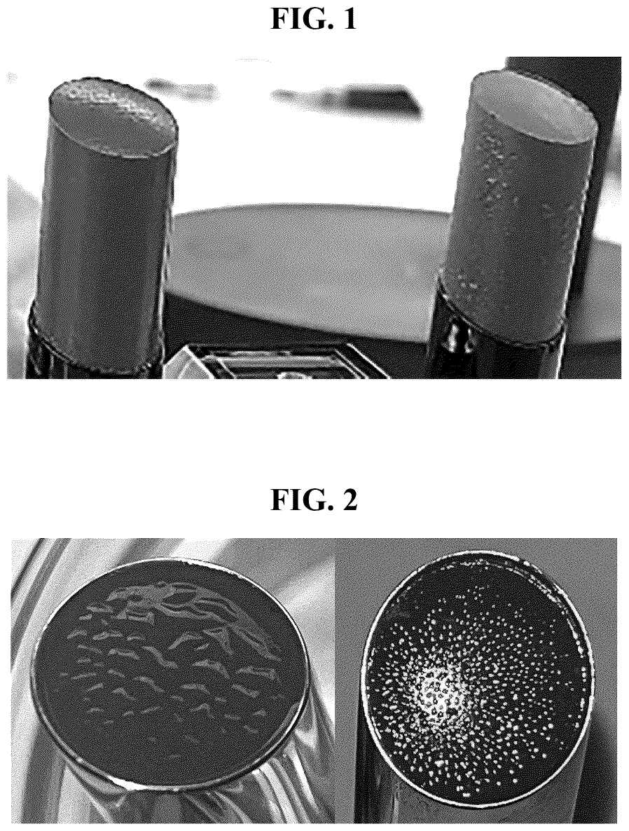

The formulation of 1-1, which does not use polyol, showed no sweating phenomenon and exhibited excellent feeling in use. The formulations of 1-2 and 1-3 contain synthetic wax and polyol. The formulation of 1-3, which uses butylene glycol as the polyol, exhibited uneven pigment dispersion and severe sweating at both a high temperature of 45° C. and room temperature. In contrast, the formulation of 1-2, which uses glycerin as the polyol, showed very low levels of sweating (see FIG. 1). Accordingly, it was found that butylene glycol has lower stability compared to glycerin in polyol-in-oil formulations. In terms of feeling in use, the formulation of 1-2 applied smoothly, providing a refreshing and moisturizing feeling in use, while the formulation of 1-3 exhibited a very light feeling in use.

In the formulations of 1-4 to 1-7, when plant-derived waxes were used instead of synthetic wax, even when glycerin was used as the polyol, severe sweating occurred, indicating significantly lower formulation stability compared to synthetic wax. In terms of feeling in use, the formulation of 1-4 showed smooth spreadability, while the formulation of 1-5 exhibited a stiff and less smooth feeling in use. The formulation of 1-6 had a low hardness, resulting in an overly soft application. Meanwhile, the formulation of 1-7 was found to fail in forming a stick. Accordingly, it was confirmed that when sunflower seed wax is used among natural waxes in the manufacture of polyol-in-oil formulations, it can form a suitable hardness for the stick formulation and provide an excellent feeling in use.

### EXAMPLE 2

The stick formulations were prepared using the same method as in Example 1, according to the formulation in Table 2, and the sweating phenomenon and feeling in use of each formulation were evaluated.

TABLE 2

| | Components | 2-1 | 2-2 | 2-3 | 2-4 | 2-5 | 2-6 | 2-7 |
|---|---|---|---|---|---|---|---|---|
| 1 | Sunflower seed wax | 10 | 10 | 10 | 10 | 10 | 10 | 10 |
| 2 | Polyglyceryl-2 triisostearate | 30 | 25 | 15 | 10 | 5 | | 10 |
| 3 | Methyl hydrogenated | 10 | 10 | 10 | 10 | 10 | 10 | 10 |
| | rosinate | | | | | | | |
| 4 | Hydrogenated poly(C6-14 | 26 | 24 | 24 | 24 | 24 | 24 | 24 |
| | Olefin) | | | | | | | |
| 5 | Diisostearyl malate | 15 | 15 | 15 | 15 | 15 | 15 | 15 |
| 6 | Cetyl PEG/PPG-10/1 | 3 | 3 | 3 | 3 | 3 | 3 | 3 |
| | dimethicone*pentaerythrityl | | | | | | | |
| | tetra-di-t-butyl | | | | | | | |
| | hydroxyhydrocinnamate | | | | | | | |
| 7 | Disteardimonium hectorite | 1 | 1 | 1 | 1 | 1 | 1 | 1 |
| 8 | Silica dimethyl silylate | | 2 | 2 | 2 | 2 | 2 | 2 |
| 9 | Pigments | 5 | 5 | 5 | 5 | 5 | 5 | 5 |
| 10 | Glycerin | | 5 | 15 | 20 | 25 | 30 | 15 |
| 11 | Butylene Glycol | | | | | | | 5 |

| | | | | | | | |
|---|---|---|---|---|---|---|---|
| 45° C. sweating | ◯ | X | X | X | Δ | ◯ | X |

It was confirmed that natural waxes, which are very susceptible to sweating compared to synthetic waxes, exhibited severe sweating even when polyol was not contained (see formulation 2-1). In contrast, when polyol was added to natural wax and silica dimethyl silylate, a porous powder, was also added, it was confirmed that sweating can be suppressed up to a polyol content of 25 wt % (see formulations 2-2 to 2-5). The formulation of 2-2, with a relatively low polyol content, showed that its moisturizing ability did not last long. The formulation of 2-3 exhibited a refresh feeling in use and long-lasting moisturizing ability, while the formulations of 2-4 and 2-5 showed a richer feeling in use and maintained long-lasting moisturizing ability compared to the formulation of 2-3. The formulation of 2-7, in which butylene glycol was additionally added, showed that when adding butylene glycol along with glycerin, the clammy (soft and sticky) feeling in use that occurs due to the increased glycerin content was improved. It was confirmed that this formulation provided a lighter and smoother application feeling in use compared to the formulation of 2-4, which contained only glycerin with a polyol content of 20 wt %. Meanwhile, the formulation of 2-6, which contained a high polyol content of 30 wt %, exhibited sweating phenomena and a clammy feeling in use.

### EXAMPLE 3

The stick formulations were prepared using the same method as in Example 1, according to the formulation in Table 3 below, and the sweating phenomenon and feeling in use of each formulation were evaluated.

TABLE 3

| | Components | 3-1 | 3-2 | 3-3 | 3-4 | 3-5 | 3-6 | 3-7 | 3-8 |
|---|---|---|---|---|---|---|---|---|---|
| 1 | Sunflower seed wax | 10 | 10 | 10 | 10 | 10 | 10 | 10 | 10 |
| 2 | Polyglyceryl-2 triisostearate | 15 | 15 | 15 | 15 | 12 | 9 | 12 | 12 |
| 3 | Methyl hydrogenated | 10 | 10 | 10 | 10 | 10 | 10 | 10 | 10 |
| | rosinate | | | | | | | | |
| 4 | Hydrogenated poly(C6-14 | 26 | 26 | 25 | 24 | 24 | 24 | 24 | 24 |
| | Olefin) | | | | | | | | |
| 5 | Diisostearyl malate | 15 | 15 | 15 | 15 | 15 | 15 | 15 | 15 |
| 6 | Cetyl PEG/PPG-10/1 | 3 | 3 | 3 | 3 | 3 | 3 | 3 | 3 |
| | dimethicone*pentaerythrityl | | | | | | | | |
| | tetra-di-t-butyl | | | | | | | | |
| | hydroxyhydrocinnamate | | | | | | | | |
| 7 | Disteardimonium hectorite | 1 | | 1 | 1 | 1 | 1 | 1 | 1 |
| 8 | Kaolin | | 1 | | | | | | |
| 9 | Silica dimethyl silylate | | | 1 | 2 | 5 | 8 | | |
| 10 | Mica | | | | | | | 5 | |
| 11 | Non-porous SILICA | | | | | | | | 5 |
| 12 | Pigments | 5 | 5 | 5 | 5 | 5 | 5 | 5 | 5 |
| 13 | Glycerin | 15 | 15 | 15 | 15 | 15 | 15 | 15 | 15 |

| | | | | | | | | |
|---|---|---|---|---|---|---|---|---|
| 45° C. sweating | ◯ | ◯ | X | X | X | X | ◯ | ◯ |

When a large amount of pigment is added to a polyol-in-oil formulation, there is an issue with poor compatibility between the pigment and polyol, resulting in uneven dispersion. In the polyol-in-oil formulation containing natural wax and polyol, the formulation of 3-1, which used distearyldimonium hectorite as the inorganic thickening agent, exhibited improved pigment dispersibility. Additionally, it prevented the contents from separating at high temperatures for extended periods after manufacturing, facilitating the stick molding process. In contrast, the formulation of 3-2, which used kaolin as the inorganic thickening agent, did not show improved pigment dispersibility. When the manufactured contents were stored at 90° C., the pigment separated within 2 hours, making it unsuitable for stick molding.

Comparing the formulations of 3-3 to 3-8, it can be observed that even with the addition of powder, the sweating phenomenon of the polyol-in-oil formulation was not improved when adding plate-like powders such as mica or non-porous silica powders. Further, when porous powder was added, it was found that the sweating phenomenon of the polyol-in-oil formulation containing natural wax can be improved. However, as the powder content increased, the spreadability became less smooth, the gloss after application disappeared, and it also caused a feeling of dryness. Therefore, to achieve excellent moisturizing ability, gloss, and to suppress sweating, it was confirmed that a powder content of 5 wt % or less is preferable.

### EXPERIMENTAL EXAMPLE 1

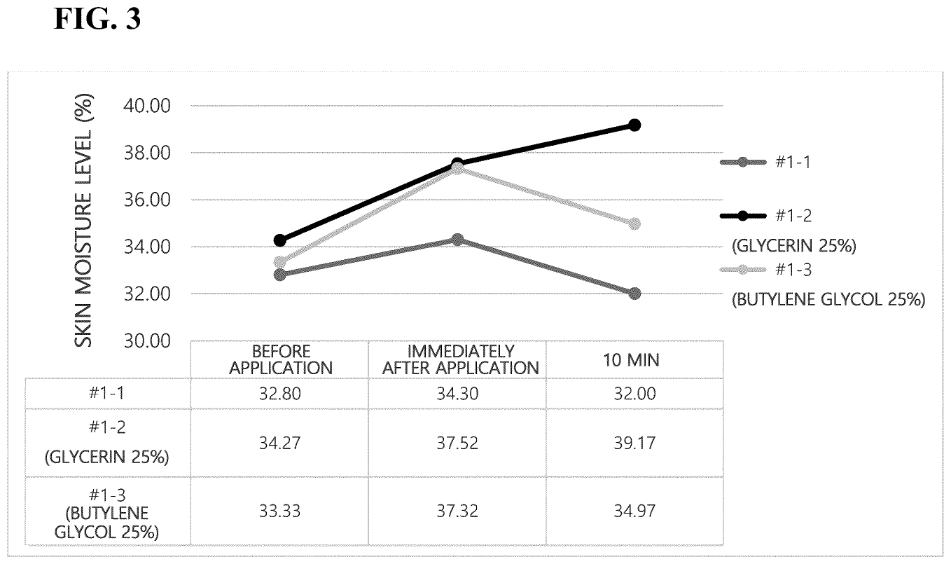

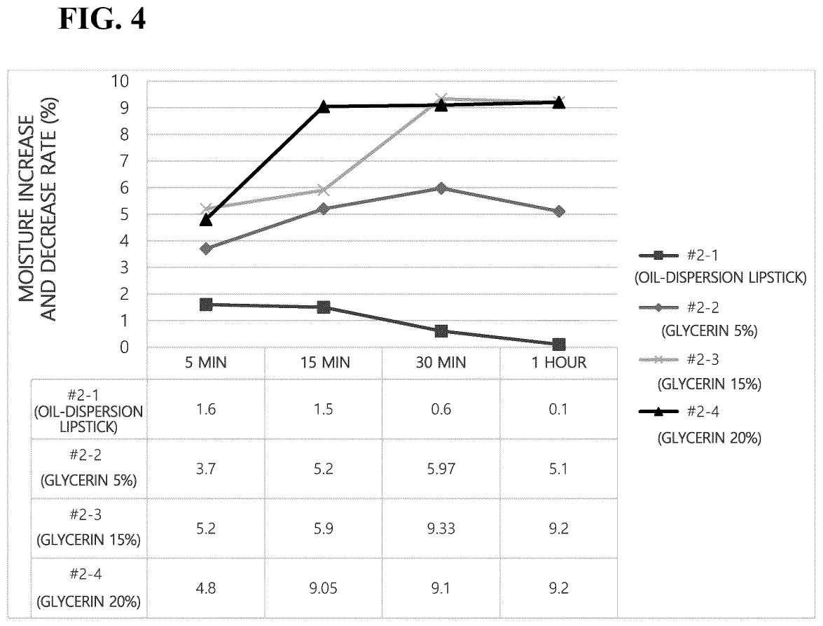

The moisturizing ability of the formulations of 1-1 to 1-3 from Example 1 and the formulations of 2-1 to 2-4 from Example 2 was evaluated based on changes in the type and content of polyol. The results are shown in FIG. 3 and FIG. 4.

The moisturizing ability was evaluated by applying 0.2 g of each sample to the forearm skin and measuring moisture levels over time using a moisture meter (Measurement device: Scalar Moisture Checker MY-808S, Scalar Corporation, Japan).

As a result, the skin moisture level immediately after application significantly increased to similar levels in both formulations of 1-2 and 1-3, which contained glycerin and butylene glycol. These formulations showed higher skin moisture levels compared to the formulation of 1-1, which did not contain polyol. Over time, the skin moisture level of the formulation 1-2 continued to increase, while the skin moisture level of the formulations 1-1 and 1-3 rapidly decreased. Accordingly, it was confirmed that glycerin not only has an excellent effect on initial moisture level but also on moisturizing durability (see FIG. 3).

Additionally, as a result of examining the moisture increase and decrease rate based on glycerin content, the initial moisture amount was higher in the order of 2-3>2-4>2-2>2-1. The formulation of 2-1 showed a rapid decrease in skin moisture amount within 1 hour, returning to a level similar to before application. At 15 minutes, the skin moisture level was higher in the formulation of 2-4, but at 30 minutes or more, there was no significant difference between the samples with 15 wt % and 20 wt % glycerin content. The formulation of 2-2 showed a decline in moisture level after 1 hour, indicating that to achieve long-lasting excellent moisturizing ability, it is preferable to use glycerin content of 10 wt % or more in polyol-in-oil formulations to differentiate from conventional products (see FIG. 4).

The above describes specific aspects of the present disclosure in detail. It will be apparent to those skilled in the art that these specific techniques are merely preferred embodiments and that the scope of the present disclosure is not limited thereby. Therefore, the substantial scope of the present disclosure is defined by the appended claims and their equivalents.

What is claimed is:

1. A cosmetic composition of a polyol-in-oil formulation, comprising wax, oil, polyol, porous powder, and an inorganic thickening agent.

2. The cosmetic composition of claim 1, wherein the wax comprises sunflower seed wax.

3. The cosmetic composition of claim 1, wherein the polyol comprises glycerin.

4. The cosmetic composition of claim 3, wherein the polyol further comprises one or more selected from the group consisting of butylene glycol, diglycerin, and propylene glycol.

5. The cosmetic composition of claim 1, wherein the porous powder comprises silica, silica silylate, silica dimethyl silylate, silica/dimethicone, silica/methicone, methyl methacrylate crosspolymer, or mixtures thereof.

6. The cosmetic composition of claim 1, wherein the inorganic thickening agent comprises one or more selected from hectorite and bentonite.

7. The cosmetic composition of claim 1, wherein the composition comprises 0.1 to 15 wt % of wax, 30 to 70 wt % of oil, 5 to 25 wt % of polyol, 0.5 to 6 wt % of porous powder, and 0.1 to 3 wt % of inorganic thickening agent, based on the total weight of the composition.

8. The cosmetic composition of claim 1, wherein the polyol and the porous powder are mixed in a weight ratio of 4 to 20:1.

9. The cosmetic composition of claim 1, further comprising 0.1 to 10 wt % of pigment, based on the total weight of the composition.

10. The cosmetic composition of claim 1, further comprising 0.1 to 5 wt % of emulsifier, based on the total weight of the composition.

11. The cosmetic composition of claim 1, further comprising 5 to 25 wt % of emollient, based on the total weight of the composition.

12. The cosmetic composition of claim 11, wherein a mixed weight of the polyol and emollient is 30 to 40 wt %, based on the total weight of the composition.

13. The cosmetic composition of claim 1, wherein the composition is a lip makeup cosmetic composition.

14. The cosmetic composition of claim 1, wherein the composition is a cosmetic composition in a stick formulation.

15. The cosmetic composition of claim 1, wherein the composition is an anhydrous formulation.
